(19) United States
(12) Patent Application Publication
(10) Pub. No.: US 2007/0016180 A1
(43) Pub. Date: Jan. 18, 2007

(54) Microwave surgical device

(76) Inventors: Fred T. Lee JR., Madison, WI (US); Christopher L. Brace, Middleton, WI (US); Paul F. Laeseke, Madison, WI (US); Daniel Warren van der Weide, Madison, WI (US)

Correspondence Address: Patula & Associates, P.C.;14th Floor, 116 S. Michigan Ave., Chicago, IL, 60603, US

(21) Appl. No.: 11/440,331

(22) Filed: May 24, 2006

Related U.S. Application Data
(63) Continuation-in-part of application No. 10/834,802, filed on Apr. 29, 2004, now Pat. No. 7,101,369.
(60) Provisional application No. 60/684,065, filed on May 24, 2005.
(60) Provisional application No. 60/690,370, filed on Jun. 14, 2005.
(60) Provisional application No. 60/702,393, filed on Jul. 25, 2005.
(60) Provisional application No. 60/707,797, filed on Aug. 12, 2005.
(60) Provisional application No. 60/710,276, filed on Aug. 22, 2005.
(60) Provisional application No. 60/710,815, filed on Aug. 24, 2005.

(51) Int. Cl. A61B 18/18

(52) U.S. Cl. 606/33

(57) ABSTRACT

A medical instrument or device used to decrease blood loss during surgery and/or other medical procedures. The device includes a microwave antenna housed in a handset (or laparoscopic probe) that is placed in close proximity to the tissue of interest. The device runs in the microwave spectrum and receives power from a from a microwave generator. When turned on (triggered), the device delivers microwave energy to tissue, providing a cutting or cautery effect.

### CLAIM OF PRIORITY

This application is a Continuation-In-Part of co-pending U.S. Non-Provisional Patent Applications entitled “Triaxial Antenna for Microwave Tissue Ablation” filed Apr. 29, 2004 and assigned U.S. application Ser. No. 10/834,802; “Segmented Catheter for Tissue Ablation” filed Sep. 28, 2005 and assigned U.S. application Ser. No. 11/237,136; “Cannula Cooling and Positioning Device” filed Sep. 28, 2005 and assigned U.S. application Ser. No. 11/237,430; and “Air-Core Microwave Ablation Antennas” filed Sep. 28, 2005 and assigned U.S. application Ser. No. 11/236,985; the entire disclosures of each and all of these applications are hereby herein incorporated by reference.

This application further claims priority to U.S. Provisional Patent Applications entitled “Segmented Catheter for Tissue Ablation” filed May 10, 2005 and assigned U.S. application Ser. No. 60/679,722; “Microwave Surgical Device” filed May 24, 2005 and assigned U.S. application Ser. No. 60/684,065; “Microwave Tissue Resection Tool” filed Jun. 14, 2005 and assigned U.S. application Ser. No. 60/690,370; “Cannula Cooling and Positioning Device” filed Jul. 25, 2005 and assigned U.S. application Ser. No. 60/702,393; “Intralumenal Microwave Device” filed Aug. 12, 2005 and assigned U.S. application Ser. No. 60/707,797; “Air-Core Microwave Ablation Antennas” filed Aug. 22, 2005 and assigned U.S. application Ser. No. 60/710,276; and “Microwave Device for Vascular Ablation” filed Aug. 24, 2005 and assigned U.S. application Ser. No. 60/710,815; the entire disclosures of each and all of these applications are hereby herein incorporated by reference.

### CROSS-REFERENCE TO RELATED APPLICATIONS

This application is related to co-pending U.S. Non-Provisional Patent Applications entitled “Triaxial Antenna for Microwave Tissue Ablation” filed Apr. 29, 2004 and assigned U.S. application Ser. No. 10/834,802; “Segmented Catheter for Tissue Ablation” filed Sep. 28, 2005 and assigned U.S. application Ser. No. 11/237,136; “Cannula Cooling and Positioning Device” filed Sep. 28, 2005 and assigned U.S. application Ser. No. 11/237,430; and “Air-Core Microwave Ablation Antennas” filed Sep. 28, 2005 and assigned U.S. application Ser. No. 11/236,985; and to U.S. Provisional Patent Applications entitled “Segmented Catheter for Tissue Ablation” filed May 10, 2005 and assigned U.S. application Ser. No. 60/679,722; “Microwave Surgical Device” filed May 24, 2005 and assigned U.S. application Ser. No. 60/684,065; “Microwave Tissue Resection Tool” filed Jun. 14, 2005 and assigned U.S. application Ser. No. 60/690,370; “Cannula Cooling and Positioning Device” filed Jul. 25, 2005 and assigned U.S. application Ser. No. 60/702,393; “Intralumenal Microwave Device” filed Aug. 12, 2005 and assigned U.S. application Ser. No. 60/707,797; “Air-Core Microwave Ablation Antennas” filed Aug. 22, 2005 and assigned U.S. application Ser. No. 60/710,276; and “Microwave Device for Vascular Ablation” filed Aug. 24, 2005 and assigned U.S. application Ser. No. 60/710,815; the entire disclosures of each and all of these applications are hereby herein incorporated by reference.

### FIELD OF INVENTION

The present disclosure relates to medical instruments for decreasing blood loss, and assisting in tissue cutting during surgery and/or other medical procedures.

### BACKGROUND

Blood loss during surgery is a substantial clinical problem. Resection of multiple tissue types in the neck, chest, abdomen, pelvis, and extremities are associated with blood loss that can be acutely life-threatening from hemodynamic effects, or if the blood loss is severe enough, can require transfusions. This can be problematic from an immunological point of view during cancer surgery. For example, increased blood loss requiring transfusions during hepatic resection increases post-resection mortality. Blood loss is also a major problem during surgery for sharp or blunt trauma, in orthopedic surgery, and in gynecologic and obstetrical procedures.

Current electrosurgical devices used for cautery and cutting, discussed below, have various associated problems and disadvantages as are known in the art. Accordingly, there is a need for a device which decreases blood loss during surgery, which overcomes the problems and disadvantages associated with current electrosurgical devices used for cautery and cutting, and which is an improvement thereover.

### SUMMARY

The device of the present disclosure is a microwave device that can be used to decrease blood loss during surgery. This device is different than electrocautery devices based on radiofrequency that are in widespread clinical use. The microwave surgical device described in this disclosure is comprised of a microwave antenna housed in a handset (or laparoscopic probe) that is placed in close proximity to the tissue of interest. When turned on (triggered), the device delivers microwave energy to tissue, providing a cautery or cutting, or combined cautery and cutting effect. Tissue can then be divided rapidly and without fear of untoward hemorrhage. This device can also be used to stop pre-existing hemorrhage on a small or large scale. For example, during open abdominal procedures, a small blood vessel can be near instantaneously cauterized by applying microwave energy directly to it.

Numerous other advantages and features of the disclosure will become readily apparent from the following detailed description, from the claims and from the accompanying drawings in which like numerals are employed to designate like parts throughout the same.

### BRIEF DESCRIPTION OF THE DRAWINGS

A fuller understanding of the foregoing may be had by reference to the accompanying drawings wherein:

FIG. 1 is an illustration of a microwave zone of ablation created using the device of the present disclosure, with 65 W applied for 2 min.

FIG. 2A is a chart illustrating the dependence of the coagulation diameter on the length of time of use of the device of the present disclosure.

FIG. 2B is a chart illustrating the dependence of the coagulation diameter on the amount of applied power during use of the device of the present disclosure.

FIG. 3 is a diagram of a delivery tool and control/feedback system for cauterizing tissue, illustrating a preferred embodiment of the present disclosure.

FIG. 4 is an illustration showing cuts and coagulation of porcine liver tissue created by the device of the present disclosure using a coaxial monopole antenna.

FIG. 5 is a schematic, cross-sectional diagram of an embodiment of an antenna and scalpel combination of the present disclosure.

FIG. 6 is a schematic diagram of an embodiment of an antenna and scissors combination of the present disclosure.

### DESCRIPTION OF DISCLOSED EMBODIMENT

While the invention is susceptible of embodiment in many different forms, there is shown in the drawings and will be described herein in detail one or more embodiments of the present disclosure. It should be understood, however, that the present disclosure is to be considered an exemplification of the principles of the invention, and the embodiment(s) illustrated is/are not intended to limit the spirit and scope of the invention and/or the claims herein.

The device of the present disclosure is different than current electrosurgical devices that are used for cautery and cutting. The disclosed device will run in the microwave (not radiofrequency) spectrum and receives power from a from a microwave generator. The preferred frequencies would be the ISM (Industrial, Scientific and Medical) bands at 915 MHz, 2.45 GHz, and 5.8 GHz, although other frequencies could also be used. Since the device is not radiofrequency based, there is no need for ground pads, and charring will not substantially affect the ability of this device to perform a cautery or cut function.

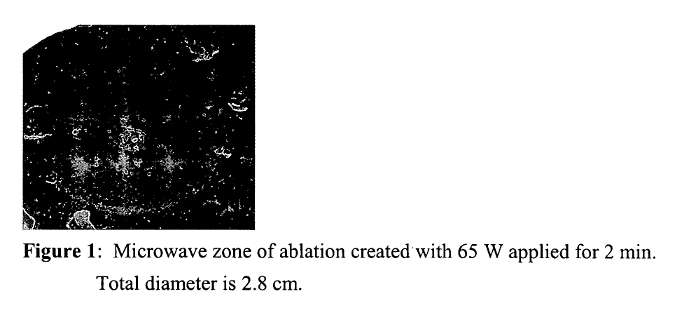

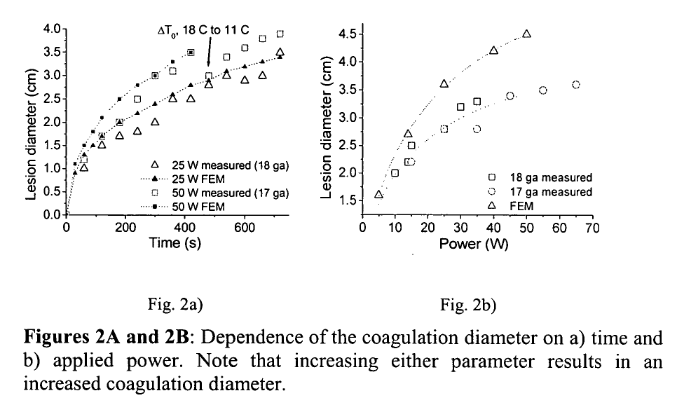

The depth of penetration of the coagulation effect can be varied depending on the amount of power that is applied, the angle at which the device is held, and the duration that the device is held in proximity to the tissue. For example, experimental data show that a region greater than 2 cm in diameter can be coagulated in 2 minutes with an input power of ˜65 W (FIG. 1). Data also shows the ablation zone diameter may be controlled by varying input power and application time (FIGS. 2A and 2B).

The specific antenna design can be variable. One possibility is to construct the microwave delivery tool based on a triaxial design, thereby taking advantage of the resonant frequency effects of triaxial catheters. However, many microwave delivery systems (e.g. coaxial near-field antennas) can be used for this purpose if they are designed to have a short protrusion of the center conductor (e.g. protrusion approximately the radius of the coaxial cable) such that in near-contact with tissue, a large absorption of microwave power is achieved.

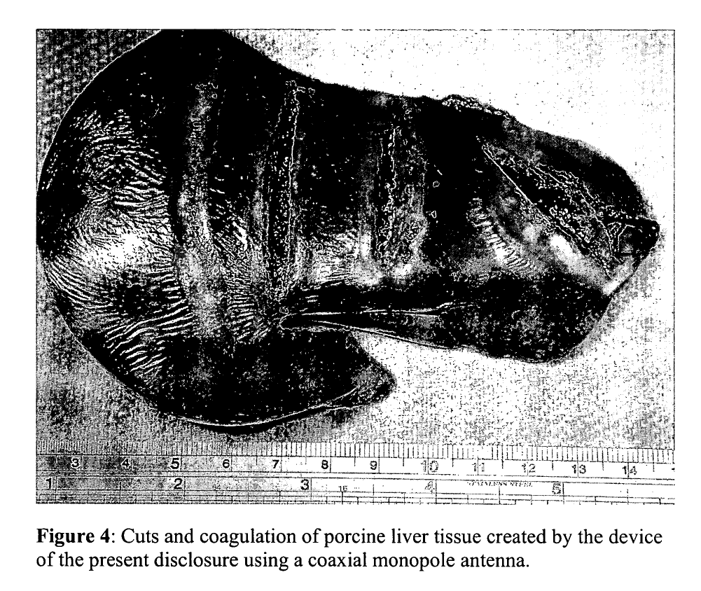

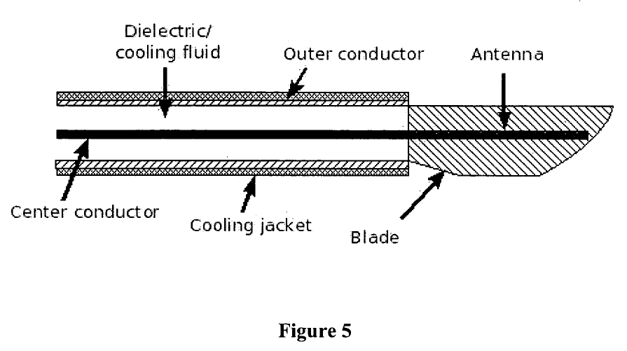

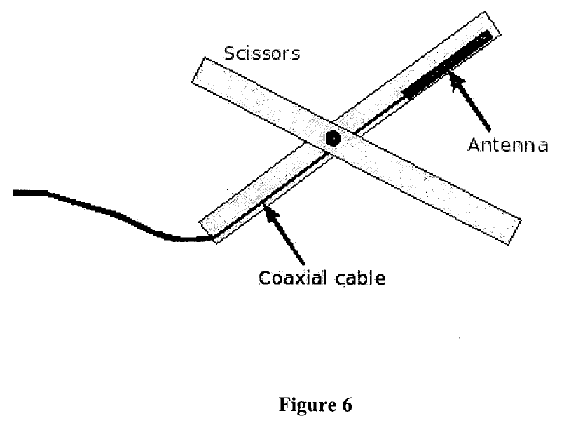

Other antenna designs may include dielectric resonators, particularly those formed in the shape of a mechanical cutting tool; coplanar, microstrip or similar waveguiding and radiating structures; spiral or helical antennas with the helix axis parallel to the coaxial feed line; planar spiral antennas; two-sided balanced or unbalanced transmission lines; antennas mounted as part of a scissors (FIG. 6), knife or scalpel (FIG. 5), clamp or other cutting or pressure-inducing device. FIG. 4 illustrates various cuts and coagulation of porcine liver tissue created by the device of the present disclosure using a coaxial monopole antenna.

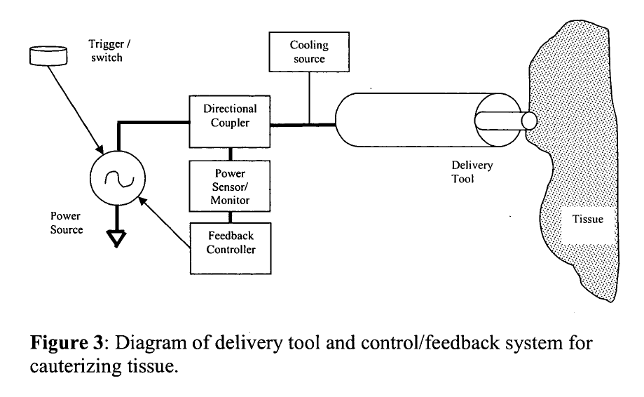

As shown in FIG. 3, the system may deliver power to the tool through a trigger switch, foot pedal or other switch or on/off button. Power reflected from the antenna can be detected and monitored to provide feedback for power control or as a safety interlock to interrupt the microwave power source if the reflected power exceeds a threshold. The control and feedback loop varies the power or duty cycle of the microwave source, enabling both safe operation and variable power application. Further, the tool can have an adjustment or calibration mechanism wherein the device can be tuned relative to the tissue of interest to a low reflected power prior to use.

The device can be mounted in a handle that is cooled by circulating fluid, gas or liquid metal. In addition, cooling fluid, gas, or liquid metal can be circulated through the center of the antenna to reduce untoward line heating as well as vary the characteristic impedance of the antenna. In one embodiment, the antenna operates at a preferential frequency of 77Ω to reduce line heating. Alternatively or in addition, the antenna can have an air-core or vacuum-core design to reduce dielectric heating. The feed of the antenna can be comprised of any conductive metal including copper, stainless steel or titanium, and the shaft can be insulated with various thermal insulators such as parylene or Teflon. The delivery tool can be coated with a biocompatible coating (e.g. a polymer such as Paralyne), and can be cooled with a water jacket.

As stated previously, this device could be used at conventional open surgery, laparoscopy, and/or percutaneously for the purpose of coagulation, vessel sealing, or cutting. The application end could house a mechanical scalpel or any other type of device to divide tissue to make an “all in one” coagulation and cutting device. The antenna could be mounted in combination with other surgical tools (one example is with a conventional scalpel), scissors, or used as a needle to stop hemorrhage. The depth of electromagnetic field penetration could be varied depending on the particular use; for example in neurosurgery, a very small amount of penetration would be desirable.

It is to be understood that the embodiment(s) herein described is/are merely illustrative of the principles of the present invention. Various modifications may be made by those skilled in the art without departing from the spirit or scope of the claims which follow.

What is claimed is:

1. A device comprising:
a tool operable in the microwave spectrum for delivering microwave energy to tissue to provide at least one of a cutting and a cautery effect.

2. A surgical device, comprising:
a microwave antenna for delivering microwave energy to tissue.

3. The device of claim 2, wherein the microwave antenna is housed in a handset.

4. The device of claim 2, wherein the microwave antenna receives power from a microwave generator.

5. The device of claim 2, wherein the microwave antenna is triaxial.

6. The device of claim 5, wherein the antenna has a length and an insertion depth, and wherein the length and insertion depth of the antenna are tunable.

7. The device of claim 2, wherein the antenna has a reflection coefficient, and wherein the reflection coefficient of the antenna is tunable.

8. The device of claim 2, wherein the microwave antenna is coaxial, and wherein a center conductor of the coaxial antenna extends from an outer conductor of the coaxial antenna.

9. The device of claim 2, wherein the microwave antenna is coplanar or constructed from coplanar waveguide or uses a coplanar waveguide feed.

10. The device of claim 2, wherein the microwave antenna is constructed from microstrip waveguide or uses a microstrip waveguide feed.

11. The device of claim 2, wherein the microwave antenna is constructed of balanced or unbalanced two-line transmission line.

12. The device of claim 2, wherein the microwave antenna is a dielectric resonator, having a blade or scalpel like shape.

13. The device of claim 2, wherein the microwave antenna is mounted as part of a clamp or pressure inducing device.

14. The device of claim 2, wherein the microwave delivery system operates at the minimum-loss characteristic impedance.

15. The device of claim 14, wherein the characteristic impedance is 77 ohms.

16. The device of claim 8, wherein the coaxial antenna includes dielectric material, and wherein the dielectric material of the coaxial delivery system is one of a fluid and a vacuum.

17. The device of claim 2, wherein at least a portion of the length of the delivery system is cooled.

18. The device of claim 17, wherein a cooling fluid circulates around the exterior of the delivery system, through a portion of the coaxial dielectric space, or through a portion of the center conductor.

19. The device of claim 2, wherein the microwave antenna is controlled through a switch mechanism.

20. The device of claim 2, wherein the microwave antenna is operatively connected to a directional coupler in combination with a power sensor and a feedback controller.

21. The device of claim 2, wherein reflected power of the microwave antenna is monitored.

22. The device of claim 21, wherein the monitored reflected power is used to control the antenna input power, application time or schedule.

23. The device of claim 21, wherein the monitored reflected power is used in an interlocking safety circuit to limit or eliminate antenna input power when a threshold reflected power is surpassed.

24. The device of claim 2, wherein the microwave antenna is mounted in combination with a scalpel, scissors or other cutting device.

25. A surgical method, comprising the steps of:
supplying power from a microwave generator to a microwave antenna; and
placing the microwave antenna in close proximity to tissue of interest to effect at least one of decreasing blood loss, coagulating blood vessels and cutting tissue.
